(19) United States
(12) Patent Application Publication
(10) Pub. No.: US 2024/0286975 A1
(43) Pub. Date: Aug. 29, 2024

(54) METHOD AND SYSTEM FOR PRODUCING HYDROCARBONS

(71) Applicant: Siemens Energy Global GmbH & Co. KG, München (DE)

(72) Inventor: Markus Kinzl, Dietzenbach (DE)

(21) Appl. No.: 18/556,119

(22) PCT Filed: Mar. 25, 2022
(86) PCT No.: PCT/EP2022/057983

§ 371 Date: May 14, 2024

(30) Foreign Application Priority Data
Apr. 30, 2021 (DE) 10 2021 204 337.2
Jun. 25, 2021 (EP) 21181829.9

(51) Int. Cl. C07C 1/24; C07C 29/151; C10J 3/00

(52) U.S. Cl. CPC C07C 1/24; C07C 29/1518; C10J 3/00; C10J 2300/0916

(57) ABSTRACT

The invention relates to a method for producing hydrocarbons including carrying out an electrolysis of water, where hydrogen and oxygen are produced and generating a carbon source, producing the hydrocarbons from the hydrogen and the carbon dioxide where at least some of the produced hydrocarbons are provided in the form of liquid hydrocarbons and an exhaust gas is formed together with the hydrocarbons. The method also includes separating the exhaust gas from the liquid hydrocarbons and recycling exhaust gas in that a reaction of the exhaust gas with the oxygen and/or water, whereby recycled products are formed which have carbon dioxide and/or carbon monoxide, water, and optionally hydrogen.

### BACKGROUND

The invention relates to a process and a plant for the production of hydrocarbons.

The characteristic of regenerative hydrocarbons is that they are produced by using regenerative starting materials. The regenerative starting materials may be, for example, regenerative carbon dioxide, which is obtained for example from biomass, and regenerative hydrogen, which is obtained for example by electrolysis of water, more particularly using power that has been generated by regenerative means. The production of the regenerative starting materials and also the reaction of the regenerative starting materials to give the regenerative hydrocarbons are energy-intensive and therefore cost-intensive as well. The costs of producing the regenerative hydrocarbons are presently several times higher than the costs of producing the same hydrocarbons from fossil raw materials.

A variety of routes exist at present that enable production of the regenerative hydrocarbons. At this point, only a brief outline will be given of the most important routes, these being Fischer-Tropsch synthesis, the alcohol-to-fuel routes, the methanol route, and the TIGAS process.

Fischer-Tropsch synthesis enables the production of various synthetic hydrocarbon fuels, more particularly diesel, kerosene, gasoline and liquefied petroleum gas. This technology, however, has two disadvantages: The first disadvantage is that the stated products are formed alongside one another and with only low selectivity, so making it necessary for all of the stated products to have to be marketed simultaneously. The desirable production of one particular product with only small amounts of the other products being formed at the same time is virtually impossible to achieve via this route. The second disadvantage of Fischer-Tropsch synthesis is that it needs carbon monoxide as a starting material. Carbon dioxide obtained regeneratively must therefore first be reduced to carbon monoxide, with the only possible ways of doing this being the two immature technologies of reverse water-gas shift (RWGS) and solid-oxide electrolysis (SOEC). The high temperatures associated with RWGS and SOEC, in the region of more than 800° C., impose extreme demands on the catalysts and materials that are needed. Under these conditions, moreover, coking of the active surfaces is likely to be comparatively rapid.

The alcohol-to-fuel routes are based on the production of ethanol by synthesis gas fermentation, in which carbon dioxide and/or carbon monoxide, and hydrogen as well, are reacted by microbiological means to give the ethanol. The ethanol is subsequently dehydrated to ethylene, which is oligomerized into oligomers. The oligomers are subsequently hydrogenated to give the hydrocarbons. In this way, a variety of the hydrocarbons can be produced with comparatively high selectivities, such as kerosene, for example. As well as the typically low space-time yield of the synthesis gas fermentation, a variety of aspects of the scale-up are viewed critically. The question therefore arises of whether, on an industrial scale, it will be possible for the alcohol-to-fuel routes to play a predominant part going forward.

The methanol route, which is based on the production of methanol from carbon dioxide and hydrogen (CO₂+3H₂⇄CH₃OH+H₂O) and/or from carbon monoxide and hydrogen (CO+2H₂⇄CH₃OH) and also the further processing of the methanol to give hydrocarbons, is more selective than Fischer-Tropsch synthesis, though less selective than the alcohol-to-fuel routes. The scale-up possibilities are estimated positively, and the possibility for direct use of regenerative carbon dioxide is a further major advantage of this route. A disadvantage in the prior art is the need for the water of reaction, formed in the methanol synthesis carried out with carbon dioxide, to be removed by energy-intensive distillation. The methanol dewatered in this way can be reacted subsequently to give various end products. Methanol-to-kerosene technology is still at the development stage (pilot plant scale). Some methanol-to-olefin technologies, aimed at the production of ethylene, propylene and butylene, have been developed to production scale. The best-known is methanol-to-gasoline technology, which has already been employed for the production of synthetic gasoline on an industrial scale. As well as gasoline as the main product, liquefied petroleum gas is formed, and, in small amounts, a C₁/C₂ hydrocarbon mixture.

### SUMMARY

Embodiments include process for producing hydrocarbons. The process includes the steps of a) performing electrolysis of water, to generate hydrogen and oxygen and b) generating a carbon source which includes carbon dioxide and carbon monoxide or consists substantially of carbon dioxide or carbon monoxide. The process also includes the step of c) producing the hydrocarbons from the hydrogen generated in step a) and the carbon source generated in step b), with at least a fraction of the hydrocarbons produced being present as liquid hydrocarbons, and with formation not only of the hydrocarbons but also of an offgas which includes hydrogen, carbon dioxide and carbon monoxide or consists substantially of hydrogen, carbon dioxide or hydrogen and carbon monoxide, where in step c), in a first process stage, methanol is produced in an equilibrium reaction, and the methanol, the hydrogen not converted in the equilibrium reaction and the carbon source not converted in the equilibrium reaction are fed to a second process stage, in which the hydrocarbons are produced from the methanol. The process further includes the steps of d) removing the offgas from the liquid hydrocarbons and e) performing offgas upgrading, in which the offgas is reacted with the oxygen generated in step a) or with the oxygen and water generated in step a) or with water, to form upgrading products which includes water, carbon dioxide and carbon monoxide or consist substantially of water and carbon dioxide or consist substantially of water and carbon monoxide, the upgrading products optionally each including hydrogen.

### BRIEF DESCRIPTION OF THE FIGURES

The invention is explained in more depth below, with reference to the schematic drawings appended, in which:

FIG. 1 shows a flow diagram of a conventional process;

FIG. 2 shows a flow diagram of the process of the invention; and

FIG. 3 shows a flow diagram of an embodiment of the process of the invention.

### DETAILED DESCRIPTION

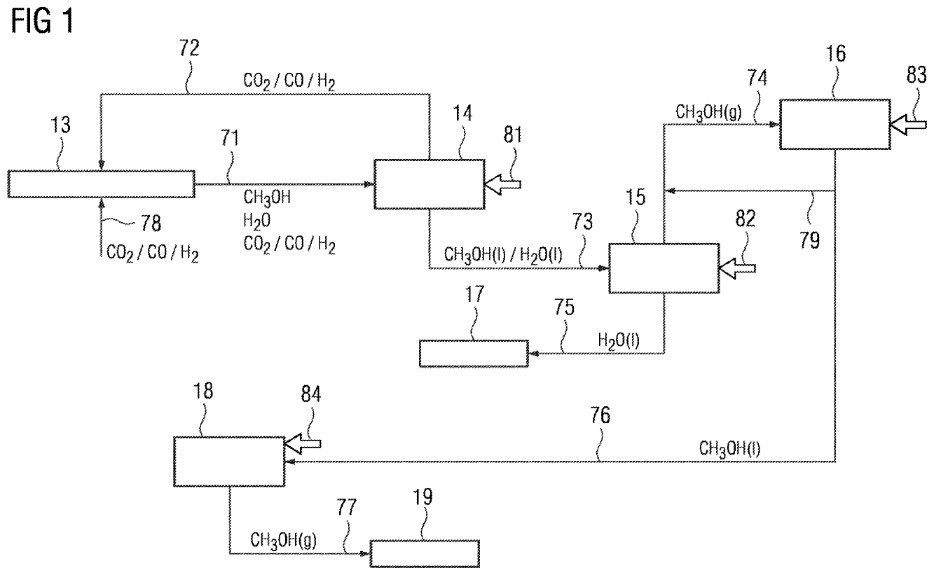

FIG. 1 represents the methanol-route process according to the prior art. A mass flow 78, which comprises carbon dioxide and/or carbon monoxide and also hydrogen or else consists of carbon dioxide and/or carbon monoxide and also hydrogen, is fed to a synthesis of methanol 13. This produces a mass flow 71 which comprises methanol, water, and unconverted reactants. Even for very high conversions of greater than 90%, the losses are not accepted. Separation 14 of the mass flow 71 is therefore carried out; in the separation 14, the water and the methanol are removed from the unconverted reactants by condensation. The unconverted reactants form a mass flow 72, which is again fed to the synthesis of methanol 13. The mixture of methanol and water formed in the separation 14 is fed in a mass flow 73 to a distillation 15, for separation of the methanol from the water. The water, in a mass flow 75, may be fed to wastewater treatment 17. The methanol has to be fed in a mass flow 74 to a condensation 16 (condenser at the top of the distillation column) and in a subsequent mass flow 76 to an evaporation 18, since the subsequent hydrocarbon synthesis 19 proceeds in the gas phase. A fraction of the condensate 16 formed in the condensation 16 may be returned to the top of the column as a mass flow 79. The separation 14 requires a first cooling 81, the distillation 15 a first heating 82, the condensation 16 a second cooling 83, and the evaporation 18 a second heating 84. This makes the operation energy-intensive and hence cost-intensive. It is necessary in spite of this to return the unconverted reactants to the mass flow 72, which makes sense when considering the high energy involved in the regenerative obtention of these reactants.

An alternative technology to the methanol-to-gasoline technology is the TIGAS process, whose first step produces a mixture containing principally methanol, dimethyl ether and water. This mixture is reacted directly, without removal of water, to give gasoline. In spite of this simplification and of various technological and economic advantages, the TIGAS technology is not presently suitable for producing regenerative hydrocarbons, since, since, similarly to Fischer-Tropsch synthesis, the TIGAS process uses carbon monoxide as a reactant (3 CO+3 H₂->CH₃—O—CH₃+CO₂), and so here as well the problem described—the relative immaturity of the RWGS technology and SOEC technology—becomes a factor.

Cost-effective production of the liquid hydrocarbons requires in all processes that the reactants employed are used as completely as possible and/or that as little energy as possible is used in the processes.

It is an object of the invention, therefore, to provide a process and a plant which allow cost-effective production of liquid hydrocarbons.

The process of the invention for producing hydrocarbons includes the steps of: a) performing electrolysis of water, to generate hydrogen and oxygen; b) generating a carbon source which includes carbon dioxide and carbon monoxide or consists substantially of carbon dioxide or carbon monoxide; c) producing the hydrocarbons from the hydrogen generated in step a) and the carbon source generated in step b), with at least a fraction of the hydrocarbons produced being present as liquid hydrocarbons, and with formation not only of the hydrocarbons but also of an offgas which includes hydrogen, carbon dioxide and carbon monoxide or consists substantially of hydrogen and carbon dioxide or hydrogen and carbon monoxide and also optionally gaseous hydrocarbons, where in step c), in a first process stage, methanol is produced in an equilibrium reaction, and the methanol, the hydrogen not converted in the equilibrium reaction and the carbon source not converted in the equilibrium reaction are fed to a second process stage, in which the hydrocarbons are produced from the methanol; d) removing the offgas from the liquid hydrocarbons; e) performing offgas upgrading, in which the offgas is reacted with the oxygen generated in step a) or with the oxygen and water generated in step a) or only with water, to form upgrading products which includes water, carbon dioxide and carbon monoxide or consist substantially of water and carbon dioxide or consist substantially of water and carbon monoxide, the upgrading products optionally each including hydrogen.

The offgas upgrading advantageously forms heat, which may likewise be upgraded, for example by converting the heat into power, for example by performing the reaction in step e) in a gas turbine, and/or by using the heat, for example to perform an evaporation and/or a distillation. Because the heat is converted into power or used, the process for producing the hydrocarbons is advantageously cost-effective. The offgas upgrading may be performed by performing combustion of the offgas with the oxygen generated in step a), which by virtue of its purity is particularly energy-rich. This has the additional advantage that the oxygen generated in the electrolysis in step a) is also upgraded, thereby making the process even more cost-effective. Alternatively, the offgas upgrading may be performed by performing gasification of the offgas by reaction with the water. The gasification may be performed without the addition of oxygen or with the addition of the oxygen generated in the electrolysis in step a). Here again, the upgrading of the oxygen in the gasification makes the process particularly cost-effective. By performing the reaction in step e) using the oxygen generated in step a) and not using air, moreover, an advantageous effect is that substantially no unwanted substances, such as nitrogen and/or noble gases, for example, are introduced into the process at this point.

In order to ensure that the fraction of the hydrocarbons produced is present in the form of the liquid hydrocarbons, the reaction products generated in step c) may be cooled. Marketing opportunities are particularly favorable for the liquid hydrocarbons obtained in that case.

In step c), in a first process stage, methanol is produced in an equilibrium reaction, and the methanol, the hydrogen not converted in the equilibrium reaction and the carbon source not converted in the equilibrium reaction are fed to a second process stage, in which the hydrocarbons are produced from the methanol. In comparison to the process of the prior art (see FIG. 1), the unreacted hydrogen and the unreacted carbon source are not returned to the synthesis of methanol (see mass flow 72). This takes away the need for energy-intensive removal of the methanol, so making the process particularly cost-effective. This is made possible by virtue of the fact that the unreacted hydrogen and the unreacted carbon source are converted into the upgrading products after the second process stage, in the offgas upgrading in step e). The two-stage process regime in step c) allows particularly efficient methanol synthesis to be achieved with the invention.

Advantageously and flexibly here, in step e), in the performance of offgas upgrading, different combinations of the reactants brought together for reaction with the offgas are possible. For instance, it is possible to bring the offgas together with the oxygen generated in step a). A further possibility is to bring together the offgas with the oxygen generated in step a) and with water, especially fresh water, for an offgas upgrading reaction. A last possibility is to perform the reaction of the offgas only with water. Alternatively or additionally to the oxygen generated, therefore, water, especially fresh water, is reacted with the offgas.

In each case, upgrading products are formed as a result that comprise water, carbon dioxide and carbon monoxide, or that consist substantially of water and carbon dioxide or substantially of water and carbon monoxide, and the upgrading products optionally each comprise hydrogen.

Different reaction pathways are therefore possible on the product side.

In one embodiment, the equilibrium reaction of the first process stage forms water, which is not removed from the methanol, from the hydrogen not converted in the equilibrium reaction and from the carbon source not converted in the equilibrium reaction. This as well makes the process particularly cost-effective. The water formed in the equilibrium reaction of the first process stage may be removed in a step g). In one embodiment, the hydrocarbons include saturated hydrocarbons and/or olefins or consist of the saturated hydrocarbons and/or the olefins. The liquid hydrocarbons, more particularly the saturated hydrocarbons, may comprise, for example, gasoline, kerosene, diesel and/or liquefied petroleum gas.

The specific reaction conditions in step c) for producing the hydrocarbons are dependent, for example, on which hydrocarbons are produced. In the production of gasoline, for example, the pressure may be from 20 bar to 30 bar, the temperature may be from 200° C. to 400° C., more particularly from 250° C. to 400° C., and a heterogeneous catalyst may be present, such as for example a zeolite catalyst, more particularly a ZSM-5 catalyst.

In one embodiment, the process includes the step of: f) removing water from the upgrading products. As a result, the upgrading products consist substantially of carbon dioxide, carbon monoxide and/or hydrogen. This gives the upgrading products a greater value than if the water were still present in the upgrading products.

The water removed in step f) is used in step a) for the electrolysis, for generating the hydrogen and the oxygen from it, and/or used in step e), for performing the reaction with the offgas. Unlike fresh water, the water removed in step f) is already demineralized. The amount of the fresh water to be fed to the process and requiring prior demineralization may therefore be reduced, so making the process even more cost-effective. Moreover, the process is consequently also suitable for performance in regions in which fresh water resources are scarce.

In one embodiment, the hydrocarbons in step c) to be also produced from the upgrading products generated in step e). This has the advantageous effect that little or no carbon is lost from the process, so making the process even more cost-effective. In one embodiment, the waters is removed in step f), since the production of the hydrocarbons is an equilibrium reaction, and water contained in the upgrading products would reduce the yield of the liquid hydrocarbons in step c).

In one embodiment, heat produced in step e) is fed entirely or partially to an endothermic step or a number of endothermic steps in the process. This allows the costs of performing the process to be reduced. The endothermic step or the endothermic steps may be, for example, an evaporation and/or a distillation.

In one embodiment, the process to be performed continuously. This produces, in particular, a continuous mass flow of the liquid hydrocarbons, a continuous mass flow of the offgas, and a continuous mass flow of the upgrading products.

In one embodiment, the fraction of the hydrocarbons produced to be present as gaseous hydrocarbons in step c) and for the reaction in step e) to be also performed with at least a fraction of the gaseous hydrocarbons produced in step c). As a result, advantageously, more heat is formed in step e) than if the reaction is performed only with carbon dioxide and/or with carbon monoxide and also with hydrogen. This is particularly relevant if not enough heat is released in step e) to maintain endothermic operations in the process. In one embodiment, the reaction in question to be gasification.

In one embodiment, in step c) the fraction of the gaseous hydrocarbons formed is a fraction of the offgas and/or for the fraction of the gaseous hydrocarbons to be removed from the liquid hydrocarbons by distillation and then admixed to the offgas.

The gaseous hydrocarbons with particular preference comprise C₁ hydrocarbon, C₂ hydrocarbon, C₃ hydrocarbon and/or C₄ hydrocarbon or with particular preference consist substantially of C₁ hydrocarbon, C₂ hydrocarbon, C₃ hydrocarbon and/or C₄ hydrocarbon. These hydrocarbons customarily have a lower commercial value than the hydrocarbons which have longer chains. The C₁ carbon and the C₂ carbon may be contained in the offgas. It is conceivable for the C₁ hydrocarbon, the C₂ hydrocarbon, the C₃ hydrocarbon and/or the C₄ hydrocarbon to be at least partly dissolved in the liquid hydrocarbons. In that case it is conceivable for the C₁ hydrocarbon and the C₂ hydrocarbon to be removed in a first distillation column and then mixed at least partly with the offgas. It is conceivable, moreover, for the C₃ hydrocarbon and the C₄ hydrocarbon to be removed in a second distillation column and then mixed at least partly with the offgas.

In step c), water is also formed, and the process includes the step of: g) removing the water from the offgas and the hydrocarbons. This may be accomplished, for example, by condensation of the water and phase separation from the liquid hydrocarbons. In one embodiment, the water removed in step g) to be used for the electrolysis in step a), for generating the hydrogen and the oxygen from it, and/or to be used in step e), for performing the reaction with the offgas. Unlike fresh water, the water removed in step g) is already demineralized. The amount of fresh water to be fed to the process and requiring prior demineralization may therefore be reduced, so making the process even more cost-effective. Moreover, the process is consequently also suitable for performance in regions in which fresh water resources are scarce. In the event of the water removed in step g) being used in step e) for performing the reaction with the offgas, moreover, the advantage arises that any hydrocarbons dissolved in the water are gasified as well.

In step b), carbon dioxide is extracted from air, biomass is burned, biomass is gasified and/or combustion offgases are generated. Processes for extracting carbon dioxide from air are known by the term “direct air capture” (DAC). For example, carbon dioxide is extracted from air via an adsorption or through a reaction with sodium hydroxide or potassium hydroxide, before it is commonly desorbed again by supply of energy and is thereby made available for subsequent operations. By the carbon dioxide being extracted from air or being obtained from the biomass, the hydrocarbons may advantageously be obtained regeneratively, i.e., without the use of fossil carbon sources. An alternative possibility is to use the combustion offgases from an unavoidable combustion operation. If the carbon dioxide is extracted from air, this has the advantage that substantially no unwanted substances, such as nitrogen and/or noble gases, for example, enter the process.

In one embodiment, the biomass to be burned or gasified by means of the oxygen generated in step a). An advantageous effect of this is that substantially no unwanted substances, such as nitrogen and/or noble gases, for example, enter the process.

The plant of the invention is configured to perform the process of the invention or an embodiment thereof.

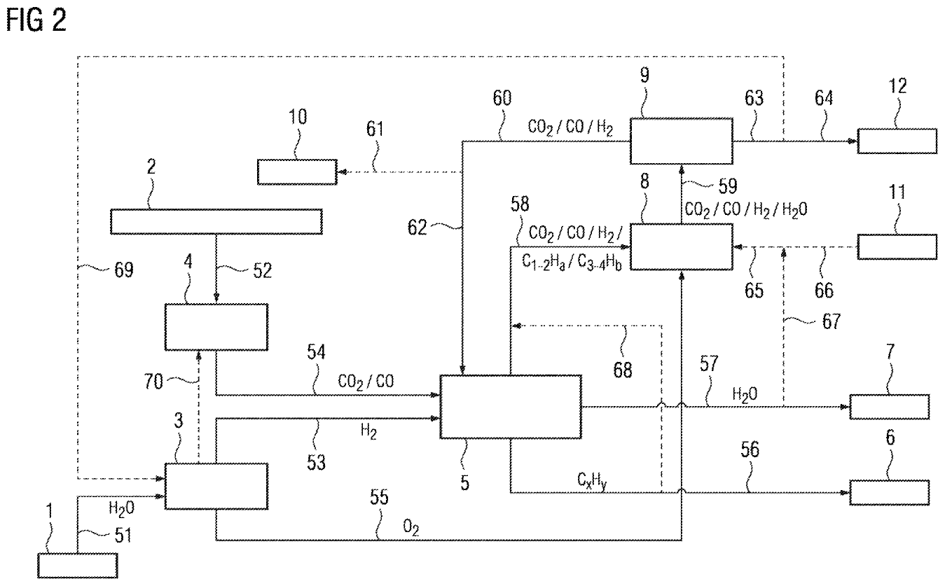

As can be seen from FIG. 2, a process for producing hydrocarbons comprises the steps of: a) performing an electrolysis 3 of water, to generate hydrogen and oxygen; b) generating a carbon source 4 which comprises carbon dioxide and/or carbon monoxide or consists substantially of carbon dioxide and/or carbon monoxide; c) producing 5 the hydrocarbons from the hydrogen generated in step a) and the carbon source generated in step b), where at least a fraction of the hydrocarbons produced are present as liquid hydrocarbons 6 and as well as the hydrocarbons 6 an offgas is also formed which comprises hydrogen, carbon dioxide and/or carbon monoxide; d) removing the offgas from the liquid hydrocarbons 6; e) performing offgas upgrading 8, in which the offgas is reacted with the oxygen generated in step a) and/or with water, to form upgrading products which comprise carbon dioxide and/or carbon monoxide, water and optionally hydrogen.

The offgas upgrading may be performed, for example, by performing combustion of the offgas with the oxygen generated in step a). In another example, the offgas upgrading may be performed by performing gasification of the offgas by reaction with the water. The gasification may be performed without the addition of oxygen or with the addition of the oxygen generated in the electrolysis in step a).

FIG. 2 shows that fresh water 1 may be fed in a mass flow 51 to the electrolysis 3. The hydrogen generated in step a) may be fed in a mass flow 53 to a reactor, in which the hydrocarbons 6 are produced in step c). The oxygen generated in step a) may be carried in a mass flow 55, separate from the mass flow 53, so that the hydrogen and the oxygen cannot mix.

To provide the carbon source in step b), a carbon-containing starting material 2 may be provided in a mass flow 52. From the mass flow 52, the carbon source may be generated, and may be fed in a mass flow 54 to the reactor. For example, in step b), carbon dioxide may be extracted from air, so that the carbon-containing starting material 2 is the air. In step b), biomass may be burned and/or gasified, so that the carbon-containing starting material 2 is the biomass. If the biomass is burned, the primary product is carbon dioxide; if the biomass is gasified, the primary product is carbon monoxide. It is conceivable for the biomass to be gasified or burned by means of the oxygen generated in step a). This has the advantage that it does not cause any nitrogen or any noble gases to enter the process. The oxygen generated in step a) may for that purpose be provided partly or completely in a mass flow 70, which is carried separately from the mass flow 53, so that the oxygen cannot mix with the hydrogen. In another example, combustion offgases may be generated, in particular by gasification and/or combustion of fossil fuels as the carbon-containing starting material.

The production 5 of the hydrocarbons may take place in step c) at a temperature in the reactor of at least 200° C., more particularly of 250° C. to 400° C. The pressure may be from 20 bar to 30 bar and there may be a heterogeneous catalyst present, such as for example a zeolite catalyst, more particularly a ZSM-5 catalyst.

In one embodiment, the offgas removed in step d) may be provided in a mass flow 58 and may include hydrogen, carbon dioxide and/or carbon monoxide. In another embodiment, the offgass removed in step d) may include gaseous hydrocarbons, especially C₁ hydrocarbon, C₂ hydrocarbon, C₃ hydrocarbon and/or C₄ hydrocarbon. In another embodiment, the offgass removed in step d) may consist substantially of hydrogen, carbon dioxide and/or carbon monoxide, or gaseous hydrocarbons, especially C₁ hydrocarbon, C₂ hydrocarbon, C₃ hydrocarbon and/or C₄ hydrocarbon.

In the event of water being also formed in step c), the process for this purpose may comprise the step of: g) removing the water 7 from the offgas and the liquid hydrocarbons 6. This produces a substantially pure mass flow 56 of the liquid hydrocarbons 6. The water 7 may be removed from the offgas by a condensation and a phase separation of the water 7. The water 7 removed in step g) may form a mass flow 57 and be used at least partly, for example, for the electrolysis 3 in step a), for generating the hydrogen and the oxygen from it. Alternatively, it is conceivable for the water 7 to be purified in a treatment plant and drained off.

In the event of the combustion or the gasification being performed in step e) with the oxygen generated in step a), the mass flow 55 may be mixed with the mass flow 58 in order to perform the offgas upgrading 8. In the event of the gasification being carried out in step e), the water 7 removed in step g) may be at least partly used in step e) for performing the reaction with the offgas-compare the mass flows 67 and 65. Alternatively or additionally to this, the gasification in step e) may be performed by means of additional fresh water 11—compare the mass flows 66 and 65.

Advantageously and flexibly, in step e), in the performance of offgas upgrading 8, various combinations of the reactants brought together for reaction with the offgas are possible. For instance, it is possible to bring the offgas together with the oxygen generated in step a). A further possibility is to bring the offgas together with the oxygen generated in step a) and with water, especially fresh water, for an offgas upgrading reaction. A final possibility is for the reaction of the offgas to be performed only with water. Alternatively or additionally to the oxygen generated, therefore, water, more particularly additional fresh water 11, is reacted with the offgas.

In each case, this forms upgrading products which comprise water, carbon dioxide and carbon monoxide, or which consist substantially of water and carbon dioxide or substantially of water and carbon monoxide, and where the upgrading products may optionally each comprise hydrogen. Different reaction pathways are therefore possible on the product side.

After step e), a mass flow 59 is formed which comprises carbon dioxide, carbon monoxide, hydrogen and/or water and/or consists substantially of carbon dioxide, carbon monoxide, hydrogen and/or water. Where combustion is carried out in step e), the carbon contained in the offgas is converted substantially into carbon dioxide. Where gasification is performed in step e), the fraction of the carbon monoxide in the mass flow 59 will be higher than the fraction of the carbon dioxide. FIG. 2 shows, moreover, that the process may comprise the step of: f) removing 9 water 12 from the upgrading products. The water 12 may be removed by condensation of the water 12. This produces a mass flow 63, formed of the water 12 removed in step f). The water 12 removed in step f) may be used in step a) for the electrolysis 3 (compare mass flow 69), for generating the hydrogen and the oxygen from it, and/or may be used in step e), for performing the reaction with the offgas. The fraction of the mass flow 63 that is not used in the mass flow 69 may form a mass flow 64. Alternatively to the use of the water 12 removed in step f), the water 12 may also be purified in a treatment plant and drained off.

FIG. 2 shows that the hydrocarbons may also be produced in step c) from the upgrading products generated in step e). For this purpose, downstream of the removal 9 of the water 12, a mass flow 60, which comprises carbon dioxide, carbon monoxide and/or hydrogen or consists substantially of carbon dioxide, carbon monoxide and/or hydrogen, may be guided in a direction toward the reactor. A fraction of the mass flow 60 may optionally be taken off in a mass flow 61 as a residual offgas 10, for any byproducts occurring to be separated out and thus prevented from accumulating in the process. A mass flow 62, formed by the mass flow 60 with the optional subtraction of the mass flow 61, is then fed to the reactor.

The process may be performed continuously. This means, for example, that the mass flows 53, 54 and 58 in particular are continuous.

In one embodiment, heat produced in step e) is fed partly or completely to an endothermic step or a plurality of endothermic steps in the process, such as an evaporation and/or a distillation, for example.

It is also conceivable for a fraction of the hydrocarbons produced to be present as gaseous hydrocarbons in step c) and for the reaction in step e) to be carried out also at least with a fraction of the gaseous hydrocarbons 6 produced in step c). In that case, in step c), the fraction of the gaseous hydrocarbons may be formed as a fraction of the offgas (this would mean that in FIG. 2, the mass flow 58 comprises the fraction of the gaseous hydrocarbons already at the start of said mass flow 58) and/or the fraction of the gaseous hydrocarbons may be removed from the liquid hydrocarbons 6 as a mass flow 68 by distillation and then admixed to the offgas, i.e., added to the mass flow 58. The gaseous hydrocarbons may comprise C₁ hydrocarbon, C₂ hydrocarbon, C₃ hydrocarbon and/or C₄ hydrocarbon or consist substantially of C₁ hydrocarbon, C₂ hydrocarbon, C₃ hydrocarbon and/or C₄ hydrocarbon. In that case, the C₁ hydrocarbon and the C₂ hydrocarbon are present preferably at the start of the mass flow 58, and the C₃ hydrocarbon and the C₄ hydrocarbon are preferably present in the mass flow 68 and are admixed to the mass flow 58 after the start thereof.

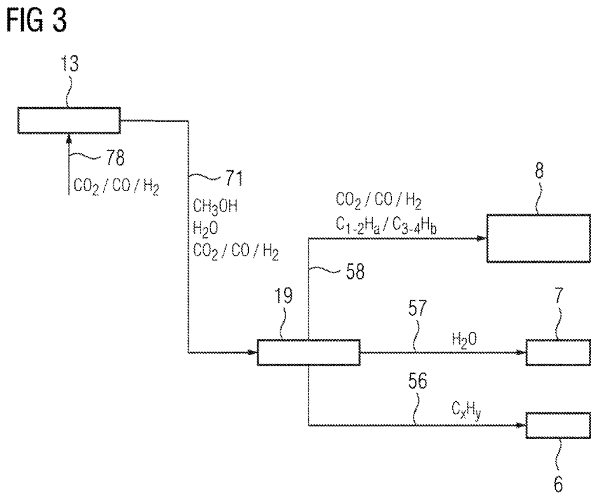

As can be seen from FIG. 3, methanol may be produced 13 in step c) in a first process stage in an equilibrium reaction, and the methanol, the hydrogen not converted in the equilibrium reaction and the carbon source not converted in the equilibrium reaction may be fed to a second process stage, in which the hydrocarbons are produced from the methanol. To produce 13 the methanol, a mass flow 78 may be provided, which comprises hydrogen and comprises carbon monoxide and/or carbon dioxide or which consists substantially of hydrogen and consists of carbon monoxide and/or carbon dioxide. In the first process stage, the reaction conditions may comprise, for example: a temperature of 200° C. to 300° C., a pressure of at least 50 bar, more particularly of 50 bar to 250 bar, and a metal oxide catalyst may be present.

As a result of production 13 of the methanol, a mass flow 71 is formed which comprises methanol and hydrogen, and also carbon monoxide and/or carbon dioxide, and optionally water. The mass flow 71 is fed as reactant to a production 19 of the hydrocarbons with methanol, i.e., is fed to the second process stage, without removal of any of the aforesaid substances. In the second process stage, for example, the pressure may be from 20 bar to 30 bar, the temperature may be from 200° C. to 400° C., more particularly from 250° C. to 400° C., and a heterogeneous catalyst may be present, such as for example a zeolite catalyst, more particularly a ZSM-5 catalyst. Steps d) and e) are performed in analogy to FIG. 1. Moreover, the offgas may also comprise C₁ to C₄ hydrocarbons.

It is conceivable for water to be formed in the equilibrium reaction of the first process stage (if the methanol is produced with carbon dioxide as reactant), this water being fed to the second process stage. The water is removed only in step d).

The process is operated with particular advantage for the production of the hydrocarbons with methanol as preferred product from a methanol synthesis 13. The methanol may be further used in turn as a valuable starting material (reactant) or as an intermediate for a production (19) of further high-value liquid hydrocarbons.

In the event that the hydrocarbons comprise C₁ hydrocarbon, C₂ hydrocarbon, C₃ hydrocarbon and/or C₄ hydrocarbon, the C₁ hydrocarbon, the C₂ hydrocarbon, the C₃ hydrocarbon and/or the C₄ hydrocarbon may for example be upgraded in a particularly simple manner if the process is performed at a site which likewise hosts a refinery. The C₁ hydrocarbon, the C₂ hydrocarbon, the C₃ hydrocarbon and/or the C₄ hydrocarbon may be used as reactants in the refinery. The refinery may comprise, for example, a steam cracker. Ethylene may be produced in the steam cracker, and has a high commercial value.

The heat produced in step e) may likewise be marketed, if the process is performed at a site which likewise hosts other chemical or pharmaceutical industry plants. The heat produced in step e) may be used, for example, to perform an endothermic operation, such as an evaporation or a distillation, for example, in the other plants. In one embodiment, a plant is configured to perform the process.

1. A process for producing hydrocarbons (6), with the steps of:
a) performing electrolysis (3) of water, to generate hydrogen and oxygen;
b) generating a carbon source (4) which comprises carbon dioxide and carbon monoxide or consists substantially of carbon dioxide or carbon monoxide;
c) producing (5) the hydrocarbons (6) from the hydrogen generated in step a) and the carbon source generated in step b), with at least a fraction of the hydrocarbons produced being present as liquid hydrocarbons, and with formation not only of the hydrocarbons (6) but also of an offgas which comprises hydrogen, carbon dioxide and carbon monoxide or consists substantially of hydrogen, carbon dioxide or hydrogen and carbon monoxide, where in step c), in a first process stage, methanol is produced (13) in an equilibrium reaction, and the methanol, the hydrogen not converted in the equilibrium reaction and the carbon source not converted in the equilibrium reaction are fed to a second process stage, in which the hydrocarbons (6) are produced from the methanol;
d) removing the offgas from the liquid hydrocarbons (6);
e) performing offgas upgrading (8), in which the offgas is reacted with the oxygen generated in step a) or with the oxygen and water generated in step a) or with water, to form upgrading products which comprise water, carbon dioxide and carbon monoxide or consist substantially of water and carbon dioxide or consist substantially of water and carbon monoxide, the upgrading products optionally each comprising hydrogen.

2. The process as claimed in claim 1, where in step c), water is formed in the equilibrium reaction and is fed to the second process stage.

3. The process as claimed in claim 1 or 2, with the step of:
f) removing (9) water (12) from the upgrading products.

4. The process as claimed in claim 3, where the water (12) removed in step f) is used in step a) for the electrolysis (3), for generating the hydrogen and the oxygen from it, and/or is used in step e), for performing the reaction with the offgas.

5. The process as claimed in any of claims 1 to 4, where in step c), the hydrocarbons (6) are also produced from the upgrading products generated in step e).

6. The process as claimed in any of claims 1 to 5, where the process is performed continuously.

7. The process as claimed in any of claims 1 to 6, where in step c), a fraction of the hydrocarbons produced are present as gaseous hydrocarbons and in step e), the reaction is also performed at least with a fraction of the gaseous hydrocarbons produced in step c).

8. The process as claimed in claim 7, where in step c) the fraction of the gaseous hydrocarbons is formed as a fraction of the offgas and/or the fraction of the gaseous hydrocarbons is removed from the liquid hydrocarbons (6) by distillation and then admixed to the offgas.

9. The process as claimed in claim 7 or 8, wherein the fraction comprises C₁ hydrocarbon, C₂ hydrocarbon, C₃ hydrocarbon and/or C₄ hydrocarbon or consists substantially of C₁ hydrocarbon, C₂ hydrocarbon, C₃ hydrocarbon and/or C₄ hydrocarbon.

10. The process as claimed in any of claims 1 to 9, where in step c) water is also formed and the process comprises the step of:
g) removing the water (7) from the offgas and the hydrocarbons (6).

11. The process as claimed in claim 10, where the water (7) removed in step g) is used in step a) for the electrolysis (3), for generating the hydrogen and the oxygen from it, and/or is used in step e), for performing the reaction with the offgas.

12. The process as claimed in any of claims 1 to 11, where in step b), carbon dioxide is extracted from air, biomass is burned, biomass is gasified and/or combustion offgases are generated.

13. The process as claimed in claim 12, where the biomass is burned or gasified by means of the oxygen generated in step a).

14. A plant configured to perform a process as claimed in any of claims 1 to 13.
